(19) United States
(12) Patent Application Publication
(10) Pub. No.: US 2023/0301320 A1
(43) Pub. Date: Sep. 28, 2023

(54) COFFEE BEVERAGE PRODUCT AND METHOD OF PREPARING THEREOF

(71) Applicant: SOCIÉTÉ DES PRODUITS NESTLÉ S.A., Vevey (CH)

(72) Inventors: EDWIN ANANTA, Epalinges (CH); DEEPAK SAHAI, Plain City, OH (US); YOUYUN LIANG, Singapore (SG)

(21) Appl. No.: 18/006,613

(22) PCT Filed: Jul. 16, 2021
(86) PCT No.: PCT/EP2021/069987

§ 371 Date: Jan. 24, 2023

Related U.S. Application Data
(60) Provisional application No. 63/056,796, filed on Jul. 27, 2020.

(51) Int. Cl. A23F 5/14; A23F 5/12; A23F 5/38; A23F 5/40

(52) U.S. Cl. CPC A23F 5/14; A23F 5/12; A23F 5/38; A23F 5/40

(57) ABSTRACT

The present invention relates to a coffee beverage product comprising 1 to 25 wt.% of coffee solids; 1-11 wt.% of milk protein; 0.01-3 wt.% galactose and 0.5-15 wt.% galactooligosaccharide. The present invention further relates to a method of preparing such a coffee beverage product.

### FIELD OF THE INVENTION

The present invention relates to a coffee beverage product beverage product with a specific combination of milk protein, sugars and galactooligosaccharide. The present invention further relates to a method of preparing such a coffee beverage product.

### BACKGROUND OF THE INVENTION

Many food products traditionally comprise high amount of sugar, which is important to achieve the sweet taste and texture the consumers expect of the product. For health reasons there is a desire to reduce the amount of sugar in many food products and to replace it with healthier ingredients, e.g. fibres, but to ensure the consumer liking of the product it is desired to achieve this without sacrificing taste and texture/mouthfeel. One solution is to use artificial sweeteners that provide sweetness without the energy contribution of sugar, but these do not contribute to the texture/mouthfeel like sugar therefore resulting in a less pleasing taste and texture/mouthfeel and furthermore many consumers want to avoid artificial ingredients such as sweeteners. Food products such as e.g. creamers, ready-to-drink or coffee mixes beverages may comprise milk, which include lactose and sucrose to achieve the desired taste and texture/mouthfeel. For the reasons given above, there is a desire to reduce the amount of sugar, preferably replacing it with healthier ingredients while preserving the taste and texture/mouthfeel of the original product.

It is known that lactose can be polymerized by beta-galactosidase to produce galactooligosaccharide (GOS). GOS acts as prebiotics and is used as an ingredient primarily in infant formulae and baby food. The yield of GOS from polymerization of lactose is normally below 50%, often in the range of 10-30%.

Any reference to prior art documents in this specification is not to be considered an admission that such prior art is widely known or forms part of the common general knowledge in the field. As used in this specification, the words “comprises”, “comprising”, and similar words, are not to be interpreted in an exclusive or exhaustive sense. In other words, they are intended to mean “including, but not limited to”.

### SUMMARY OF THE INVENTION

An object of the present invention is to improve the state of the art and to provide an improved solution to overcome at least some of the inconveniences described above or at least to provide a useful alternative. The object of the present invention is achieved by the subject matter of the independent claims. The dependent claims further develop the idea of the present invention.

Accordingly, the present invention provides in a first aspect a coffee beverage product comprising 1 to 25 wt.% of coffee solids; 1-11 wt.% of milk protein; 0.01-3 wt.% galactose and 0.5-15 wt.% galactooligosaccharide; all weight percentages on a dry basis.

In a second aspect, the invention relates to a method of preparing a coffee beverage product according to the present invention.

It has been found by the inventors that a coffee beverage product with a specific combination of milk protein, sugars and GOS allows replacing skim milk powder or other milk ingredients as well as part of the sucrose normally added while achieving similar taste and texture/mouthfeel. The coffee beverage advantageously has reduced total sugar content and at the same time provides prebiotic GOS.

### DETAILED DESCRIPTION OF THE INVENTION

As used in the specification, the word “comprise”, “comprising” and the like are to be construed in an inclusive sense as opposed to an exclusive or exhaustive sense; that is to say, in the sense of “including”, but not “limited to”.

As used in the specification, the word “about” should be understood to apply to each bound in a range of numerals. Moreover, all numerical ranges should be understood to include each whole integer within the range.

As used in the specification, the singular forms “a”, “an” and “the” include plural referents unless the context clearly dictate otherwise.

Unless noted otherwise, all percentage in the specification refer to weight percent, where applicable.

Unless defined otherwise, all technical and scientific terms have and should be given the same meaning as commonly understood by one of the ordinary skill in the art which this invention belongs.

The term dry weight refers to the measurement of the mass of matter when completely dried and all fluids removed form the matter. The dry weight % of a substance refers to the relative amount of said substance in the total dry matter. As used in the specification, the expression “weight %” and “wt%” are synonymous.

It is noted that the various features, aspects, examples and embodiments described in the present invention may be compatible and/or combined together.

Other definitions and/or explanations are present in the detailed description below.

### Definitions

By beta-galactosidase is understood one or more enzymes with enzymatic activity of enzyme class EC 3.2.1.23, also called beta-D-galactoside galactohydrolase, exo-(1->4)-beta-D-galactanase or lactase, which catalyses the hydrolysis of terminal non-reducing beta-D-galactose residues in beta-D-galactosides, as well as transgalactosylation by transferring a galactose moiety of a beta-D-galactoside to another sugar molecule.

EC (Enzyme Committee) numbers refer to the definition of enzymatic activity and nomenclature given by the Nomenclature Committee of the International Union of Biochemistry and Molecular Biology as in force on 3rd July 2019.

Galactooligosaccharides (GOS) are oligosaccharides composed of different galactosyl residues (usually from 2 to 9 units) and a terminal glucose linked by β-glycosidic bonds, such as β-(1-2), β-(1-3), β-(1-4), and β-(1-6). GOS are normally produced through the enzymatic conversion of lactose.

The present invention relates in part to a coffee beverage product comprising 1 to 25 wt.% of coffee solids; 1-11 wt.% of milk protein; 0.01-3 wt.% galactose and 0.5-15 wt.% galactooligosaccharide; all weight percentages on a dry basis.

A coffee beverage product is a beverage product in powder or liquid form comprising coffee solids. The powdered coffee beverage product is usually reconstituted in hot or cold liquid before consumption, while the liquid coffee beverage product can either be consumed directly without addition of liquid or diluted in liquid before consumption if it is on the form of a concentrated liquid coffee beverage product.

The coffee beverage product of the present invention comprises 1 to 25 wt.% of coffee solids. The coffee solids comprised in the beverage product of the present invention may be in any suitable form, e.g. in the form of roast and ground coffee, powdered dried water extract of roast and ground coffee, for example freeze-dried or spray-dried coffee powder (i.e. soluble coffee powder), a coffee extract or coffee concentrate obtained by the extraction of roast and ground coffee beans or a combination thereof. In one embodiment of the present invention, the coffee beverage product comprises 1 to 20 wt.% of coffee solids. In another embodiment, the coffee beverage product comprises 1 to 15 wt.% of coffee solids. In a preferred embodiment, the coffee beverage product of the present invention comprises 1 to 10 wt.% of coffee solids.

In an embodiment, the coffee beverage product of the present invention comprises 1-11 wt% milk protein. By milk protein is meant any protein or combination of proteins derived from milk. The milk proteins may have been derived from milk in any suitable way. In another embodiment, the coffee beverage product comprises 2-10 wt% milk protein, for example 3-9 wt% milk protein. In yet another embodiment, the coffee beverage product comprises 5-10 wt% milk protein, for example 6-9 wt% milk protein.

The carbohydrates (or sugars) comprised in the coffee beverage product of the present invention is primarily galactooligosaccharide (GOS). GOS is preferably resulting from the enzymatic treatment of milk with beta-galactosidase. The enzymatic treatment has the effect of reducing lactose in milk by partly converting the sugar into GOS. The end products of the enzymatic reaction of milk are GOS, glucose, galactose and residual lactose. Therefore, in one embodiment, the coffee beverage product of the present invention comprises 0.01-3 wt.% galactose and 0.5-15 wt.% galactooligosaccharide. In another embodiment, the coffee beverage product comprises 0.02-3 wt.% galactose and 0.7-14 wt.% galactooligosaccharide.

Advantageously, the coffee beverage product of the present invention has then reduced amount of total sugar and contains beneficial prebiotic GOS while maintaining similar sweetness perception.

In one embodiment, the coffee beverage product of the present invention comprises 0.01-4.5 wt.% lactose and 0.01-3 wt.% glucose. The lactose and glucose are preferably resulting from the enzymatic treatment of milk by beta-galactosidase.

In one embodiment, the coffee beverage product may comprise sucrose. Due to the amounts of lactose, glucose and GOS present in the product, the amount of sucrose may be reduced as compared to a conventional coffee beverage product while retaining an acceptable taste, sweetness and texture. In another embodiment, the coffee beverage product of the invention does not comprise sucrose.

The coffee beverage product of the invention may comprise a foamer ingredient. By foamer ingredient is understood an ingredient which when dissolved in an aqueous liquid can produce a foam on top of a beverage product. The foam may be produced by any suitable mechanisms, such as e.g. by a chemical reaction between the foamer ingredient and the liquid that produces a gas, or by release of gas held in pores in the foamer ingredient into the liquid when the foamer ingredient is dissolved.

In one embodiment, the coffee beverage product of the invention is a ready-to-drink product or a powdered coffee mix product.

Another aspect of the invention relates to a method for preparing a beverage coffee product of the present invention comprising the steps of providing a milk powder enzymatically treated with beta-galactosidase containing and mixing said milk powder with coffee solids.

To perform the enzymatic treatment of the milk, an aqueous composition comprising milk powder, for example skimmed milk and/or whole milk, reconstituted in water is first provided. For example, the aqueous composition comprises 30-80 wt% of total solid content.

A beta-galactosidase is then added to the aqueous composition. The beta-galactosidase may be any suitable beta-galactosidase. The amount of beta-galactosidase added may depend on the specific enzyme, its level of activity and its formulation. The skilled person will know how to choose the amount according to the desired result. Typically, the amount will be in the order of 0.5-2% by weight of lactose in the aqueous composition. The aqueous composition may be at the desired reaction temperature when the beta-galactosidase is added, or it may be heated to the reaction temperature following the addition of the beta-galactosidase. In this step, the beta-galactosidase is allowed to react with the lactose in the aqueous composition to hydrolyse the lactose and produce GOS. The enzyme is allowed to react for 10-240 minutes at 50-65° C. It has been found that this temperature range facilitates the hydrolysis of lactose and formation of GOS.

After the beta-galactosidase has been allowed to react, the composition is heat treated at 70-150° C. for 10-150 seconds, preferably at 72-130° C. for 10-120 seconds. The purpose of the heat treatment is to prevent substantial degradation of the GOS by further reaction of the beta-galactosidase, as well as ensuring the microbiological stability of the product, without creating excessive denaturation of protein or formation of Maillard reaction products. The milk treated with the enzyme may be used directly as it is after the heat treatment, or it may e.g. be dried to obtain a powder, for example by spray-drying, packed, stored and/or distributed for subsequent use.

In one embodiment, the milk enzymatically treated with beta-galactosidase contains 2-5 wt% lactose; 10-13 wt% glucose; 1-4 wt% galactose; and 28-33 wt% galactooligosaccharide.

In one embodiment, glucose, lactose, galactose and GOS are preferably derived from the enzymatic treatment of milk with beta-galactosidase.

Those skilled in the art will understand that they can freely combine all features of the present invention disclosed herein. In particular, features described for the product of the present invention may be combined with the method of the present invention and vice versa. Further, features described for different embodiments of the present invention may be combined. Where known equivalents exist to specific features, such equivalents are incorporated as if specifically referred to in this specification.

Further advantages and features of the present invention are apparent from the figures and non-limiting examples.

### EXAMPLES

### Example 1 Method of Treating Enzymatically Skimmed Milk Powder

Skimmed milk powder (MSK) was mixed with water to form a slurry with 60% total solids content at 60° C. 1% of a beta-galactosidase with transgalactosylase activity was added to the MSK slurry. The reaction was allowed to proceed for duration of 60 min. Pasteurization at 85° C. for 18 seconds was applied to pasteurize the milk and partially inactivate enzymes before spray-drying to form dried skimmed milk powder. It was demonstrated that with spray drying, it was possible to completely inactivate the enzymes and no residual enzyme activity was detected in the final spray dried powder. The composition of the skimmed milk powder and the enzyme treated powder is shown in Table 1.

TABLE 1

Composition of skimmed milk powder (MSK) before and after enzymatic treatment with beta-galactosidase

| % by weight | MSK before treatment | MSK after treatment |
|---|---|---|
| Glucose | 0 | 12.41 |
| Lactose | 53 | 2.47 |
| Galactose | 0 | 2.9 |
| GOS | 0 | 33 |
| Total sugars | 53 | 17.78 |
| Sugar reduction | NA | 66.5% |

The carbohydrate analysis of the skimmed milk powder after enzymatic treatment shows that galactooligosaccharide (GOS) forms most of the polysaccharide and that the total sugar content is dramatically reduced.

### Example 2 Coffee Mix Compositions

Different coffee mix compositions were produced by mixing soluble coffee powder with standard skimmed milk powder (i.e. no treatment with the enzyme; Sample 1) or skimmed milk powder after enzymatic treatment (i.e. skimmed milk with reduced total sugar content; Sample 2). A third coffee mix composition containing added sucrose (sweetened recipe; Sample 3) was also produced.

| | Sample 1 -Unsweetened foaming recipe with MSK after enzymatic treatment (reference) | Sample 2 -Unsweetened non-foaming recipe with MSK after enzymatic treatment | Sample 3 -Sweetened non-foaming recipe with standard MSK |
|---|---|---|---|
| Soluble coffee powder | 2 g | 2 g | 2 g |
| Skimmed Milk powder (std or enzymatically treated) | 8 g | 8 g | 8 g |
| Foamer ingredient | 2 g | 0 g | 0 g |
| Sucrose | 0 g | 0 g | 9 g |
| Total | 12 g | 10 g | 19 g |

The different samples (12 g of Sample 1; 10 g of Sample 2; 12 g of Sample 3) were reconstituted in 180 ml hot water and tasted by a trained panel of 12 tasters.

Despite the reduction of total sugar, and due to the presence of glucose and galactose, the coffee mix composition according to the present invention (Sample 2) was not perceived as less sweet when compared to standard recipe (Sample 1) containing lactose as the only sugar. This demonstrates that the significant reduction in sugars in the final recipe does not impact the sweetness level. Only Sample 3 was perceived as significantly sweeter, as expected.

### Example 3 Ready-to-Drink Coffee Composition

Two ready-to-drink samples were produced by first dry mixing the different ingredients and then adding water. The samples were tasted by a trained panel of 9 tasters.

| | Standard recipe with standard MSK (% w/w) | Recipe with MSK after enzymatic treatment (% w/w) |
|---|---|---|
| Soluble coffee powder | 2.1 | 2.1 |
| Skimmed Milk powder | 6.9 | 6.9 |
| Sodium bicarbonate | 0.15 | 0.15 |
| Sugar | 4.1 | 3.82 |
| Avacil | 0.4 | 0.4 |
| Water | 86.39 | 86.6 |
| Total | 100 | 100 |

Although the ready-to-drink coffee product of the present invention had about 10% total sugar reduction, the tasters rated similar sweetness levels when compared to standard ready-to-drink coffee product.

1. A coffee beverage product comprising 1 to 25 wt.% of coffee solids; 1-11 wt.% of milk protein; 0.01-3 wt.% galactose and 0.5-15 wt.% galactooligosaccharide; all weight percentages on a dry basis.

2. A coffee beverage product of claim 1 comprising 0.01-4.5 wt.% lactose and 0.01-3 wt.% glucose.

3. The coffee beverage product of claim 1 comprising a foamer ingredient.

4. The coffee beverage product of claim 1 wherein the product is a ready-to-drink product or a powdered coffee mix product.

5. The coffee beverage product of claim 1 wherein the coffee solids is powdered dried water extract of roast and ground coffee.

6. A method of preparing a beverage coffee product comprising 1 to 25 wt.% of coffee solids; 1-11 wt.% of milk protein; 0.01-3 wt.% galactose and 0.5-15 wt.% galactooligosaccharide; all weight percentages on a dry basis comprising the steps of providing a milk enzymatically treated with beta-galactosidase and mixing said treated milk powder with coffee solids.

7. A method according to claim 6 wherein the milk enzymatically treated with beta-galactosidase contains 2-5 wt% lactose; 10-13 wt% glucose; 1-4 wt% galactose; and 28-33 wt% galactooligosaccharide; all weight percentages on a dry basis.

8. A method according to claim 6 wherein the milk is whole milk or skimmed milk.

9. A method according to claim 6 wherein the milk enzymatically treated with beta-galactosidase is in the form of a powder.
